(19) United States
(12) Patent Application Publication
(10) Pub. No.: US 2026/0364570 A1
(43) Pub. Date: Nov. 19, 2026

(54) METHOD FOR PREPARING 2-METHYLTETRAHYDROFURAN USING MIXTURE OF COMPOUNDS DERIVED FROM LIGNOCELLULOSIC BIOMASS, CATALYST USED THEREFOR, AND METHOD FOR PREPARING THE CATALYST

(71) Applicant: IUCF-HYU (INDUSTRY-UNIVERSITY COOPERATION FOUNDATION HANYANG UNIVERSITY), Seoul (KR)

(72) Inventors: Jeong Gil SEO, Seoul (KR); Youngbin KIM, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 19/719,391

(22) Filed: Jun. 25, 2026

Related U.S. Application Data
(63) Continuation of application No. PCT/KR2024/018607, filed on Nov. 22, 2024.

(30) Foreign Application Priority Data
Dec. 29, 2023 (KR) 10-2023-0197083

(51) Int. Cl. B01J 23/755; B01J 21/04; B01J 21/06; B01J 35/61; B01J 35/63; B01J 35/64; B01J 37/00; B01J 37/02; B01J 37/03; B01J 37/04; B01J 37/08; B01J 37/18; C07D 307/08

(52) U.S. Cl. CPC B01J 23/755; B01J 21/04; B01J 21/066; B01J 35/613; B01J 35/615; B01J 35/633; B01J 35/647; B01J 37/0036; B01J 37/0201; B01J 37/0236; B01J 37/036; B01J 37/04; B01J 37/088; B01J 37/18; C07D 307/08

(57) ABSTRACT

The present invention relates to a method for preparing 2-methyltetrahydrofuran using a mixture of compounds derived from lignocellulosic biomass, a catalyst used therefor, and a method for preparing the catalyst. According to the present invention, 2-methyltetrahydrofuran can be prepared in a high yield from a mixture of compounds derived from lignocellulosic biomass by performing a hydrogenation and dehydration reaction using a furfural compound and a levulinic acid compound as reactants, and the above effect can be maximized through optimization of the composition of the reactants and reaction conditions. In addition, selectivity of 2-methyltetrahydrofuran can be further improved by using a catalyst in which nickel and copper, which are non-noble metal materials, are supported on an acid support in the reaction.

### CROSS-REFERENCE TO RELATED APPLICATIONS

This application is a continuation of International Application No. PCT/KR2024/018607 filed on Nov. 22, 2024, which claims priority to Korean Patent Application No. 10-2023-0197083 filed on Dec. 29, 2023, the entire contents of which are herein incorporated by reference.

### TECHNICAL FIELD

The present invention relates to a method for preparing 2-methyltetrahydrofuran using a mixture of compounds derived from lignocellulosic biomass, a catalyst used therefor, and a method for preparing the catalyst, and more particularly, to a method for preparing 2-methyltetrahydrofuran by reacting a furfural compound and a levulinic acid compound, which are compounds derived from lignocellulosic biomass, a catalyst used therefor, and a method for preparing the catalyst.

### BACKGROUND ART

Lignocellulosic biomass refers to a resource derived from herbaceous plants, and comprises cellulose and hemicellulose as main components. The lignocellulosic biomass can be converted into various industrial chemicals capable of replacing fossil fuels through chemical reactions, and thus has been attracting attention as a resource for producing chemical raw materials for carbon reduction.

One example of a substance that can be synthesized using lignocellulosic biomass as a raw material is 2-methyltetrahydrofuran (MTHF). 2-Methyltetrahydrofuran can be used for various purposes such as a solvent for oil extraction and organic chemical reactions, an electrolyte, and a gasoline additive. For example, Korean Patent Publication No. 10-2000-0055681, which describes a polymer blend electrolyte and an electrochemical cell using the same, discloses that 2-methyltetrahydrofuran can be used as an organic electrolyte.

As such, 2-methyltetrahydrofuran is a versatile material, and when 2-methyltetrahydrofuran is synthesized from a compound derived from biomass, it can be utilized as a high value-added compound. However, since a wide variety of intermediates are generated in the synthesis reaction of 2-methyltetrahydrofuran, there has been a limitation in that it is difficult to set appropriate reaction environments and conditions.

Accordingly, research has been conducted to increase selectivity in the synthesis of 2-methyltetrahydrofuran, and a representative method involves using a catalyst. For example, U.S. Pat. No. 5,883,266 relates to a hydrogenated 5-carbon compound and a method for preparing the same, and discloses that a high-yield reaction can be carried out when a catalyst prepared using palladium and rhenium is used. However, the above technology has a problem in that economic efficiency is poor because a catalyst comprising a noble metal as a main component is used.

Accordingly, there is a need for development of a technology capable of preparing 2-methyltetrahydrofuran in a high yield by an economical and simple method.

### SUMMARY

### Technical Problem

One object of the present invention is to provide a method for preparing 2-methyltetrahydrofuran (MTHF) in a high yield from a compound derived from lignocellulosic biomass.

Another object of the present invention is to provide a catalyst capable of improving selectivity of MTHF in preparing 2-methyltetrahydrofuran.

Still another object of the present invention is to provide a method for preparing the catalyst.

### Technical Solution

In order to achieve the above objects, the present invention provides a method for preparing 2-methyltetrahydrofuran, comprising obtaining 2-methyltetrahydrofuran (MTHF) by subjecting a furfural compound and a levulinic acid compound to hydrogenation and dehydration reactions.

In the present invention, a volume ratio of the furfural compound and the levulinic acid compound may be 1:5 to 10:1.

In the present invention, the reaction may be performed under a pressure of 10 to 50 bar.

In the present invention, the reaction may be performed under a temperature condition of 150 to 400 °C.

In the present invention, a solvent used in the reaction may comprise at least one selected from the group consisting of ethanol, propanol, butanol, toluene, cyclopentyl methyl ether (CPME), gamma-valerolactone (GVL), and methyl isobutyl ketone (MIBK).

In the present invention, the reaction may be performed in the presence of a catalyst comprising an acid support, and nickel metal and copper metal supported on the acid support.

In the present invention, the reaction may be performed in a batch reactor or a continuous reactor.

In the present invention, the method for preparing 2-methyltetrahydrofuran may be performed by a two-step continuous reaction comprising converting a part of levulinic acid into gamma-valerolactone in a lignocellulosic biomass comprising furfural and levulinic acid; and obtaining 2-methyltetrahydrofuran by reacting the furfural and remaining levulinic acid.

The present invention also provides a catalyst for preparing 2-methyltetrahydrofuran, comprising an acid support, and nickel metal and copper metal supported on the acid support.

In the present invention, the acid support may comprise at least one selected from the group consisting of aluminum oxide, zirconium oxide, zinc oxide, titanium oxide, magnesium oxide, cerium oxide, and lanthanum oxide.

The present invention also provides a method for preparing the catalyst for preparing 2-methyltetrahydrofuran.

The method for preparing a catalyst of the present invention may comprise mixing a nickel salt and a copper salt with an acid support and stirring; and forming a catalyst in which nickel and copper are supported on the acid support by drying, calcining, and reducing the mixture for which the stirring has been completed.

In the present invention, the acid support may be prepared through forming a sol by stirring a metal salt solution and adding an acid; obtaining a xerogel by drying the sol; and obtaining an acid support by pulverizing and calcining the xerogel.

In the present invention, the metal salt solution may comprise aluminum alkoxide and zirconium alkoxide in a weight ratio of 1:1 to 10:1.

In the method for preparing a catalyst of the present invention, a drying process may be performed at a temperature of 60 to 150 °C.

In the method for preparing a catalyst of the present invention, a calcining process may be performed at a temperature of 350 to 600 °C.

### Advantageous Effects

According to the present invention, by performing a hydrogenation and dehydration reaction using a furfural compound and a levulinic acid compound as reactants, 2-methyltetrahydrofuran can be prepared in a high yield from a mixture of compounds derived from lignocellulosic biomass, and the above effect can be maximized through optimization of the composition of the reactants and reaction conditions. In addition, selectivity of 2-methyltetrahydrofuran can be further improved by using a catalyst having nickel and copper, which are non-noble metal materials, supported on an acid support in the reaction.

### DESCRIPTION OF DRAWINGS

FIG. 1 illustrates a reaction pathway of a hydrogenation and dehydration reaction according to an embodiment of the present invention.

FIG. 2 illustrates a catalyst synthesis process using a wet impregnation method according to an embodiment of the present invention.

FIG. 3 illustrates a catalyst support synthesis process using a sol-gel method according to an embodiment of the present invention.

FIG. 4 illustrates a nitrogen adsorption isotherm of a catalyst prepared using a wet impregnation method according to an embodiment of the present invention.

FIG. 5 illustrates an XRD pattern of a catalyst prepared using a wet impregnation method according to an embodiment of the present invention.

FIG. 6 illustrates a TEM image of a catalyst prepared using a wet impregnation method according to an embodiment of the present invention.

FIG. 7 illustrates a nitrogen adsorption isotherm of a catalyst prepared using a sol-gel method according to an embodiment of the present invention.

FIG. 8 illustrates an XRD pattern of a catalyst prepared using a sol-gel method according to an embodiment of the present invention.

FIG. 9 illustrates a TEM image of a catalyst prepared using a sol-gel method according to an embodiment of the present invention.

FIG. 10 illustrates an ammonia temperature-programmed desorption analysis result of a catalyst prepared using a sol-gel method according to an embodiment of the present invention.

FIG. 11 is a graph illustrating selectivity of products according to reaction time in a synthesis reaction of 2-methyltetrahydrofuran according to an embodiment of the present invention.

### DETAILED DESCRIPTION

Hereinafter, specific embodiments of the present invention will be described in more detail. Unless otherwise defined, all technical and scientific terms used herein have the same meaning as commonly understood by a person skilled in the art to which the present invention belongs. In general, the nomenclature used herein is well known and commonly used in the art.

The present invention relates to a method for preparing 2-methyltetrahydrofuran (MTHF) from a chemical substance derived from lignocellulosic biomass, and a catalyst used therefor.

2-Methyltetrahydrofuran is a material used as a solvent for oil extraction and organic chemical reactions or as a gasoline additive, and when MTHF is synthesized from a compound derived from biomass, it can be utilized as a high value-added compound. However, since a wide variety of intermediates are generated in the MTHF synthesis reaction, there has been a limitation in that it is difficult to set appropriate reaction environments and conditions.

The present invention is intended to overcome such limitations of the prior art, and in the present invention, selectivity of MTHF can be improved by combining a furfural compound and a levulinic acid compound and using the same as reactants in the synthesis of 2-methyltetrahydrofuran. In addition, a conversion rate to MTHF can be increased by using, as a catalyst, a catalyst in which nickel and copper, which are non-noble metal materials, are supported on an acid support.

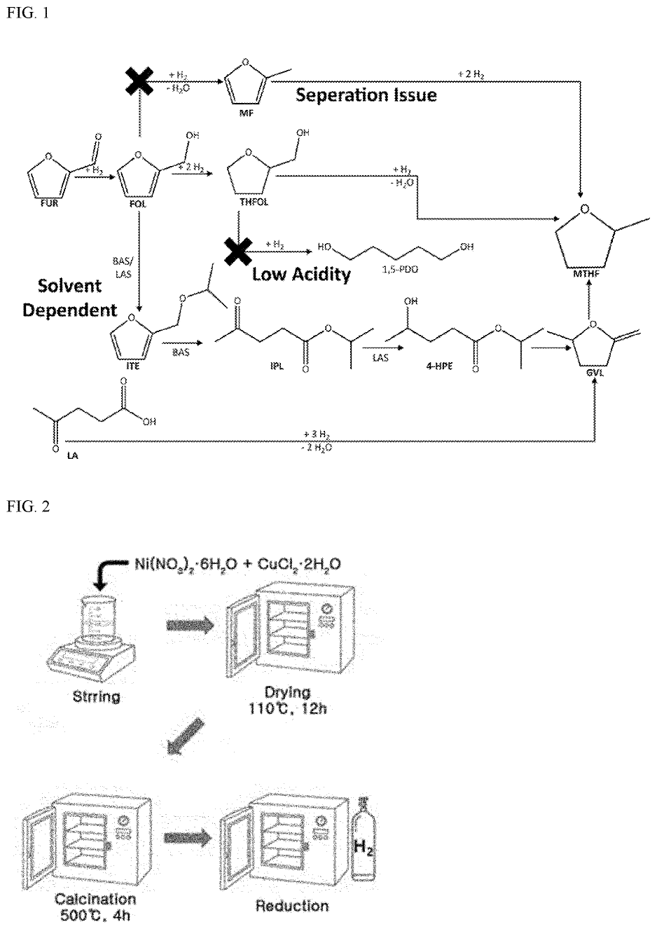

Specifically, in the present invention, as shown in FIG. 1, a furfural (FUR) compound and a levulinic acid (LA) compound are combined and reacted to undergo several chemical reactions including hydrogenation and dehydration. In this process, a yield of MTHF can be improved by generating tetrahydrofurfuryl alcohol (THFOL) and gamma-valerolactone (GVL), which are reaction intermediates capable of being further converted to 2-methyltetrahydrofuran (MTHF), and the yield of MTHF can be maximized through optimization of the composition of the reactants and reaction conditions. In addition, since furfural and levulinic acid used in the present invention are materials derived from hemicellulose and cellulose, respectively, lignocellulosic biomass can be used in its entirety in the reaction of the present invention.

Accordingly, the present invention can provide a method for preparing 2-methyltetrahydrofuran by subjecting a furfural compound and a levulinic acid compound to hydrogenation and dehydration reactions. In the present invention, the furfural compound, the levulinic acid compound, and the 2-methyltetrahydrofuran compound are interpreted as including each substance and derivatives thereof.

In the present invention, by using a furfural compound and a levulinic acid compound in combination as reactants for MTHF synthesis, a significantly improved effect on selectivity of MTHF can be achieved compared to when each of the compounds is used alone.

In the present invention, the furfural compound and the levulinic acid compound may be mixed in a volume ratio of 1:5 to 10:1, preferably 2:3 to 8:1, and more preferably 3:2 to 5:1. Within the above range, the effect of improving MTHF selectivity by using the furfural compound and the levulinic acid compound in combination is maximized, so that MTHF can be obtained in a high yield.

In relation thereto, in the Examples of the present invention, reactions were performed by adjusting the volume ratio of furfural and levulinic acid to 1:4, 2:3, 1:1, 3:2, 4:1, and 7:1, respectively, under the same conditions, and as a result, it was confirmed that when the ratio of furfural and levulinic acid was 4:1, the proportion of GVL was greatly reduced and the selectivity of MTHF was the highest.

In the present invention, the reaction pressure may be 10 to 50 bar, preferably 20 to 45 bar, and more preferably 25 to 40 bar. By adjusting the reaction pressure as such, the conversion rate to MTHF can be improved. In relation thereto, in the Examples of the present invention, the reaction pressure was adjusted to 10, 20, and 30 bar, respectively, under the same conditions, and as a result, it was confirmed that the selectivity of MTHF was the highest under a condition of 30 bar.

In the present invention, the reaction temperature may be 150 to 400 °C, preferably 180 to 350 °C, and more preferably 220 to 300 °C. By adjusting the reaction temperature within the above range, the conversion rate to MTHF can be improved. In relation thereto, in the Examples of the present invention, the reaction temperature was adjusted to 150, 200, and 250 °C, respectively, under the same conditions, and as a result, it was confirmed that the selectivity of MTHF was the highest at 250 °C.

In the present invention, the reaction may be performed in a solution phase. In this case, at least one solvent such as ethanol, propanol, butanol, toluene, cyclopentyl methyl ether (CPME), gamma-valerolactone (GVL), and methyl isobutyl ketone (MIBK) may be used as a reaction solvent, and it is preferable to use propanol or toluene.

In relation thereto, in the Examples of the present invention, 2-propanol, toluene, cyclopentyl methyl ether (CPME), gamma-valerolactone (GVL), or methyl isobutyl ketone (MIBK) was used as a reaction solvent, and as a result, it was confirmed that when toluene was used, the MTHF conversion rate was the highest at 72.0%, when 2-propanol was used, the conversion rate was high at 55.0%, whereas when CPME, MIBK, and GVL were used, the conversion rates were low at 39.0, 22.0, and 18.0%, respectively.

In the present invention, the reaction may be performed in a batch reactor or a continuous reactor.

The batch reactor is a device using a method of mixing and reacting raw materials and then releasing them at once, and the continuous reactor is a reactor using a method of continuously feeding raw materials, stirring, and continuously releasing after completion of the reaction. The reaction of the present invention shows a higher conversion rate to MTHF when performed in a batch reactor, but even when a continuous reactor is used, the effect of improving MTHF selectivity by using furfural and levulinic acid in combination can be confirmed.

When the reaction is performed in the continuous reactor, a weight hourly space velocity (WHSV) of a feedstock may be adjusted to 0.05 to 1 mL/min, preferably 0.1 to 0.5 mL/min.

In an embodiment of the present invention, the reaction may be performed as a two-step continuous reaction. The two-step continuous reaction may be performed through converting a part of levulinic acid into gamma-valerolactone in a lignocellulosic biomass comprising furfural and levulinic acid; and synthesizing MTHF by reacting the furfural and remaining levulinic acid.

Since the amount of levulinic acid derived from cellulose derived from the initial lignocellulosic biomass is greater than the amount of furfural derived from hemicellulose, in order to optimize the ratio of furfural and levulinic acid, a part of levulinic acid is first converted into gamma-valerolactone, which can be used as a reaction solvent, as described above, and then the reaction is performed. This enables efficient preparation of MTHF using lignocellulosic biomass having a high proportion of levulinic acid.

When MTHF is prepared using a furfural compound and a levulinic acid compound as reactants according to the present invention, the yield of MTHF can be increased compared to when they are used alone, and the selectivity of MTHF can be greatly improved through adjustment of the ratio thereof and reaction conditions.

In addition, selectivity of MTHF can be further improved by performing the reaction in the presence of a bimetallic catalyst.

Accordingly, the present invention can also provide a catalyst used in hydrogenation and dehydration reactions in preparing 2-methyltetrahydrofuran (MTHF).

The catalyst for preparing MTHF of the present invention comprises an acid support, and nickel metal and copper metal supported on the acid support, and when the catalyst is applied to the MTHF synthesis reaction, selectivity to MTHF can be improved. The catalyst of the present invention can be prepared through a simple impregnation method, and since an acid support and non-noble metal materials are used, economic efficiency is secured, stability is excellent, and handling of raw materials for catalyst preparation is easy.

In the present invention, the acid support of the catalyst is a metal oxide, and may comprise, for example, at least one of aluminum oxide, zirconium oxide, zinc oxide, titanium oxide, magnesium oxide, cerium oxide, lanthanum oxide, and the like.

In the present invention, the acid support preferably comprises aluminum oxide. In relation thereto, in the Examples of the present invention, aluminum oxide and zirconium oxide were used as acid supports of the catalyst, respectively, and as a result, it was confirmed that the effect of improving MTHF conversion rate of the catalyst prepared using an aluminum oxide support was much more excellent.

In an embodiment of the present invention, the acid support may comprise aluminum oxide and zirconium oxide. In this case, the conversion rate to MTHF can be further increased through adjustment of the ratio of the aluminum oxide and the zirconium oxide.

In the present invention, by using nickel and copper as components supported on the acid support, selectivity of MTHF can be improved by a synergistic effect of nickel and copper.

In relation thereto, in the Examples of the present invention, it was confirmed that when a bimetallic catalyst comprising both nickel and copper was used, the conversion rate to MTHF increased compared to a catalyst prepared by supporting nickel and copper separately on the same acid support.

In the present invention, nickel and copper may be comprised in an amount of 10 to 40 wt %, preferably 15 to 30 wt %, based on the total weight of the catalyst, and in this case, a weight ratio of nickel and copper may be 4:1 to 1:4, preferably 2:1 to 1:2.

In the present invention, selectivity of MTHF in the hydrogenation and dehydration reaction for MTHF synthesis can be effectively improved through the interaction of nickel and copper in the catalyst and the use of the acid support, and the selectivity of MTHF can be maximized through adjustment of the type and composition of the support.

Therefore, the catalyst of the present invention can be usefully used in the MTHF synthesis reaction using hydrogenation and dehydration reactions. When the catalyst is applied to the method for preparing MTHF of the present invention, the catalyst may be used in an amount of 0.05 to 1 g, preferably 0.1 to 0.3 g, per 1 mL of reactant.

The catalyst for preparing MTHF of the present invention can be prepared through an impregnation method. Specifically, the catalyst of the present invention can be prepared through mixing a nickel salt and a copper salt with an acid support and stirring; and forming a catalyst in which nickel and copper are supported on the acid support by drying, calcining and reducing the mixture for which the stirring has been completed.

The nickel salt serves as a precursor of nickel supported on the catalyst, and at least one selected from the group consisting of nickel nitrate, nickel sulfate, nickel chloride, and nickel carbonate may be used, and may be used in a hydrate form.

The copper salt serves as a precursor of copper supported on the catalyst, and at least one selected from the group consisting of copper chloride, copper nitrate, copper sulfate, and copper carbonate may be used, and may be used in a hydrate form.

The acid support is commercially available or can be prepared using a sol-gel method.

Specifically, when the acid support comprises two or more metal oxides, the acid support can be prepared through forming a sol by stirring each metal salt solution and adding an acid; obtaining a xerogel by drying the sol; and pulverizing and calcining the xerogel. In the preparation method, the xerogel refers to a dry gel from which liquid has been removed, and as the metal salt, an alkoxide salt of a metal, for example, an ethoxide, propoxide, butoxide, or the like of a metal may be used.

As an example, when preparing a catalyst comprising aluminum oxide and zirconium oxide as the acid support, the weight ratio of aluminum alkoxide and zirconium alkoxide in the metal salt solution may be adjusted to 1:1 to 10:1, preferably 2:1 to 6:1, and specifically 3:1 to 5:1. When the catalyst prepared by adjusting the ratio of aluminum oxide and zirconium oxide in the acid support as described above is used, hydrogenation and dehydration reactions can be effectively promoted and selectivity of MTHF can be improved.

In relation thereto, in the Examples of the present invention, an experiment was conducted by adjusting the weight ratio of aluminum isopropoxide and zirconium butoxide in preparing an acid support comprising both aluminum oxide and zirconium oxide, and as a result, it was confirmed that a catalyst comprising an acid support prepared using about 11 g of zirconium alkoxide relative to 42 g of aluminum alkoxide had a higher conversion rate to MTHF than a catalyst using an aluminum oxide alone support. Through this, it was confirmed that the selectivity of MTHF can be maximized by mixing aluminum oxide and zirconium oxide as an acid support and adjusting the ratio thereof.

The acid support, nickel salt, and copper salt are mixed in a solution phase. In this case, the solvent of the solution may be at least one selected from the group consisting of water (distilled water), acetone, alcohol, ethyl acetate, hexane, heptane, toluene, dichloromethane, tetrahydrofuran, acetonitrile, dimethylacetamide, dimethylformamide, dimethyl sulfoxide, dimethyl oxalate, and methylpyrrolidone, and is preferably distilled water. In addition, the stirring of the solution may be performed for 1 to 24 hours, preferably 6 to 18 hours.

When the mixed solution is prepared, a step of drying the mixed solution to obtain a solid sample is performed.

The drying may be performed using known techniques and devices such as an oven, hot air, constant temperature and humidity, and microwave, and may be performed at a temperature of 60 to 150 °C, preferably 100 to 120 °C, in order to completely remove the solvent.

After evaporating all the solvent through the drying process, a step of calcining the solid sample at a high temperature is performed. The calcining may be performed at a condition of 350 to 600 °C, preferably 400 to 550 °C, while increasing the temperature. In this case, a temperature increase rate may be 2 to 8 °C/min, preferably 4 to 6 °C/min, and a total calcining time may be 2 to 6 hours, preferably 3 to 5 hours. Thereafter, the catalyst can be obtained through a process of reducing at 400 to 800 °C for 30 minutes to 5 hours.

The catalyst prepared as described above has a structure in which nickel and copper are supported on an acid support, and selectivity of MTHF in the MTHF synthesis reaction can be effectively improved according to the interaction of nickel and copper in the catalyst and the adjustment of acid sites according to the composition of the acid support.

### EXAMPLES

Hereinafter, the present invention will be described in more detail through Examples. However, these Examples are presented to illustrate some experimental methods and configurations for exemplarily explaining the present invention, and the scope of the present invention is not limited to these Examples.

### Preparation Example 1: Preparation of Catalyst Using Nickel, Copper, and Aluminum Oxide

According to the method shown in FIG. 2, a NiCu/Al₂O₃ catalyst was prepared by a wet impregnation method using nickel and copper, which are non-noble metal materials, and aluminum oxide.

10 g of Al₂O₃ used as a support and 100 mL of distilled water were stirred at 350 rpm, and simultaneously, 6.2 g of nickel nitrate hexahydrate (Ni(NO₃)₂·6H₂O) at 10 wt % and 5.45 g of copper chloride dihydrate (CuCl₂·2H₂O) were dissolved and stirred for 12 hours. The stirred solution was covered with aluminum foil with holes made therein, and then dried at 110 °C for 12 hours. The dried sample was calcined at 500 °C for 4 hours at a ramping rate of 5 °C/min. Thereafter, reduction was performed at 600 °C for 2 hours using a hydrogen 10%/nitrogen mixed gas at a flow rate of 30 mL/min at a ramping rate of 10 °C/min to obtain a NiCu/Al₂O₃ catalyst.

### Preparation Example 2: Preparation of Catalyst Using Nickel, Copper, and Aluminum Oxide/Zirconium Oxide

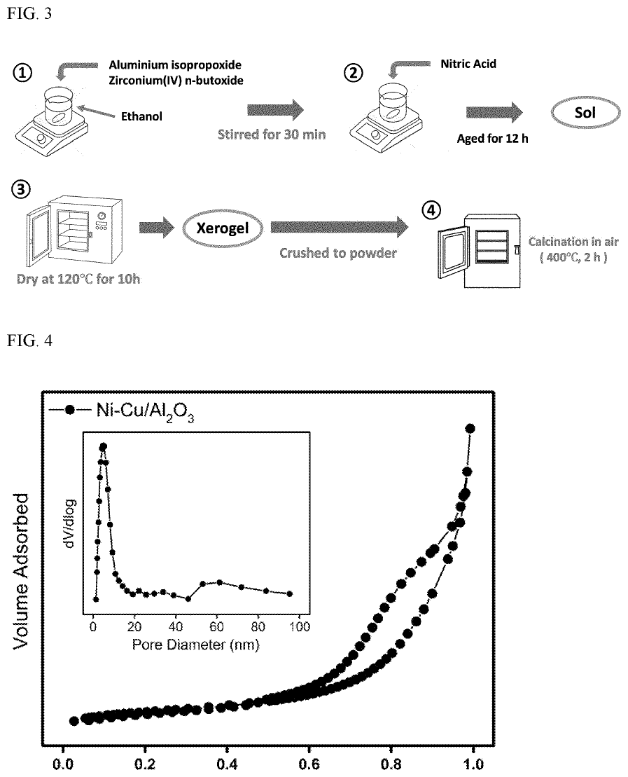

According to the method shown in FIG. 3, an acid support was prepared by a sol-gel method using Al₂O₃ and ZrO₂, and a catalyst was prepared using the same.

4.2 g of aluminum isopropoxide was dissolved in 250 mL of ethanol, and then 102.9 mL of zirconium (IV) n-butoxide (1.049 g/mL) was added and stirred for 30 minutes. 13.5 mL of nitric acid was slowly added little by little to the stirred solution while stirring, and after the addition was completed, the solution was aged at room temperature for 12 hours. The sol formed by the aged solution was dried at 120 °C for 10 hours to form a xerogel. The xerogel was finely ground into powder and then calcined at 300 °C for 2 hours to obtain an Al₂O₃_ZrO₂ support.

Using the method of Preparation Example 1, but using the Al₂O₃_ZrO₂ support instead of Al₂O₃, a NiCu/Al₂O₃_ZrO₂ catalyst was prepared. The catalyst prepared by the above method is denoted as NiCu/Al₂O₃_ZrO₂ (1), and in the following Experimental Examples, the ratio of Al₂O₃ and ZrO₂ in the acid support was adjusted by multiplying Al₂O₃ by n and ZrO₂ by 1/n according to the value (n) indicated in parentheses based thereon.

### Synthesis Example 1: MTHF Synthesis Reaction Using Batch Reactor

A reaction was performed in a batch reactor using furfural and levulinic acid as reactants and 2-propanol as a reaction solvent. 1 g of catalyst per batch was used, and the solvent and reactants were used in a ratio of 45 mL of solvent and 5 mL of reactants.

Before starting the reaction, an inert gas substitution process remaining inside the reactor was performed through a purging process using argon gas (4 bar). Thereafter, the reactor was filled with hydrogen gas (20 bar), and the reaction was performed at 200 °C at 500 rpm. The product obtained from the disassembled reactor after completion of the reaction was filtered to remove impurities, and then analyzed by HPLC (High Performance Liquid Chromatography) (Shimadzu SCL-40).

RID-20A was used as an HPLC analysis detector, PL-1170-6830 from Agilent was used as a column, and HPLC Water/0.005 M H₂SO₄ was used as an analysis solvent.

### Synthesis Example 2: MTHF Synthesis Reaction Using Continuous Reactor

A reaction was performed in a continuous reactor using furfural and levulinic acid as reactants and 2-propanol as a reaction solvent. The solvent and reactants were used in a ratio of 45 mL of solvent and 5 mL of reactants.

The continuous reactor was configured in a three-layer structure, with 8 g of glass beads placed in the lower part, 5 g of NiCu/Al₂O₃ catalyst in the center, and 3 g of glass beads in the upper part. At this time, WHSV was set to 2 (0.2 mL/min) to perform the reaction, reduction was performed once more at 400 °C for 1 hour using hydrogen, and the reaction was performed at 250 °C under a condition of hydrogen 30 bar for 12 hours. The product after the reaction was analyzed by HPLC under the same conditions and environment as the batch reactor.

### Experimental Example 1: Analysis of Physicochemical Properties of Catalyst

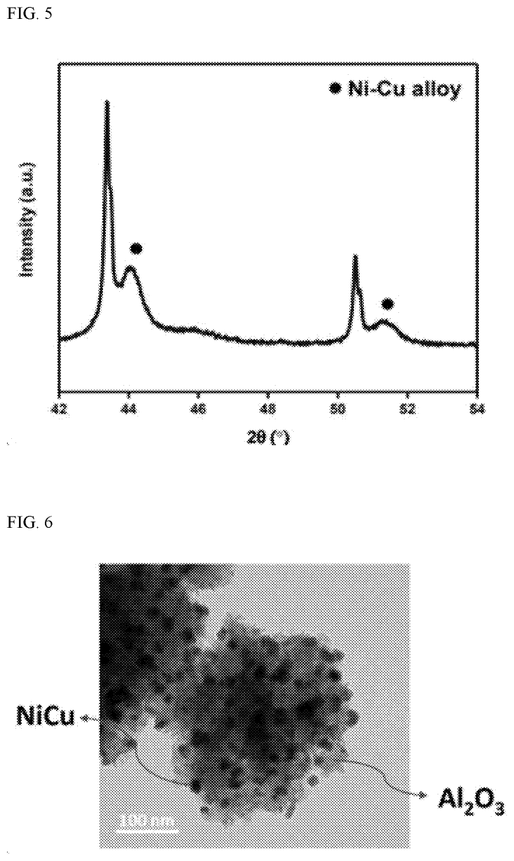

The surface area, pore volume, and pore diameter of the catalyst prepared according to the method of Preparation Example 1 were measured, and the results are shown in Table 1 below. In addition, physicochemical properties were analyzed through nitrogen adsorption isotherms, XRD analysis, and TEM images of FIGS. 4 to 6.

TABLE 1

| | Surface | Pore | Pore |
|---|---|---|---|
| Catalyst | area (m²/g) | volume (cm³/g) | diameter (nm) |
| NiCu/Al₂O₃ | 137.5 | 0.39 | 5.2 |

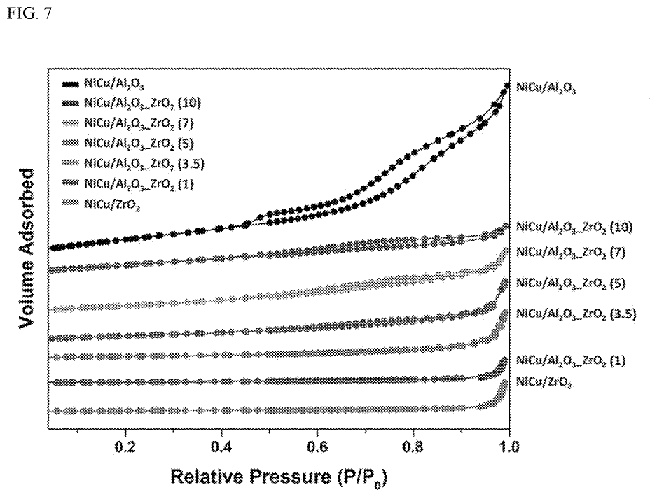

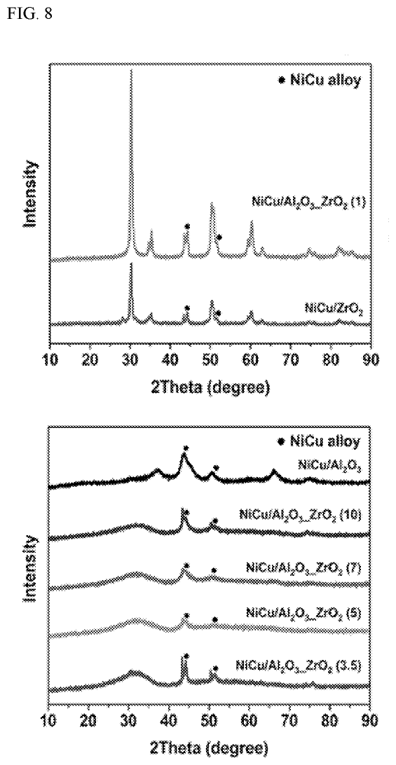

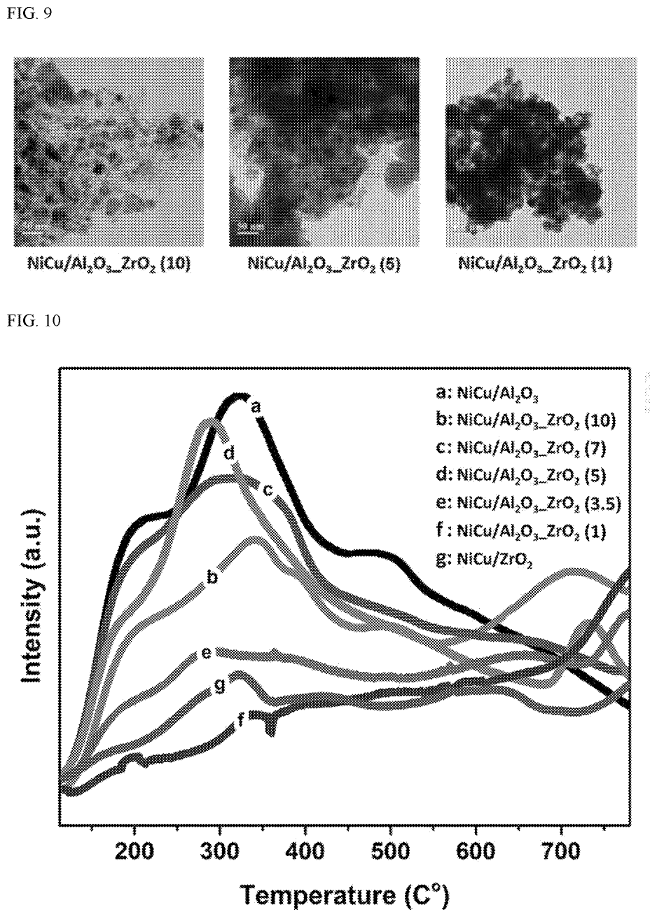

In addition, the surface area, pore volume, and pore diameter of the catalysts prepared according to the method of Preparation Example 2 with the compositions shown in Table 2 below were measured, and the results are shown together in Table 2. In addition, physicochemical properties were analyzed through nitrogen adsorption isotherms, XRD analysis, and TEM images of FIGS. 7 to 9.

TABLE 2

| | Surface | Pore | Pore |
|---|---|---|---|
| Catalyst | area (m²/g) | volume (cm³/g) | diameter (nm) |
| NiCu/Al₂O₃ | 139 | 0.26 | 7.36 |
| NiCu/Al₂O₃_{_{—}}ZrO₂ (10) | 136 | 0.19 | 5.59 |
| NiCu/Al₂O₃_{_{—}}ZrO₂ (7) | 113 | 0.23 | 8.19 |
| NiCu/Al₂O₃_{_{—}}ZrO₂ (5) | 75.8 | 0.20 | 10.95 |
| NiCu/Al₂O₃_{_{—}}ZrO₂ (3.5) | 30.8 | 0.15 | 19.95 |
| NiCu/Al₂O₃_{_{—}}ZrO₂ (1) | 11.8 | 0.08 | 27.01 |
| NiCu/ZrO₂ | 10.8 | 0.10 | 36.34 |

As a result of the experiment, the surface area of the NiCu/Al₂O₃ catalyst was 137 m²/g, which is higher than 100 m²/g, whereas in the NiCu/Al₂O₃_ZrO₂ catalyst using the Al₂O₃_ZrO₂ support, the surface area decreased as the content of ZrO₂ increased.

In addition, it was confirmed through the nitrogen adsorption isotherms that a catalyst having a mesoporous form corresponding to Type IV was synthesized, and in the XRD (X-ray Diffraction) pattern, it was confirmed that the catalyst was successfully synthesized as a Ni—Cu alloy peak that can be visually confirmed was generated after supporting nickel and copper metals on the Al₂O₃ and Al₂O₃_ZrO₂ supports.

Additionally, as a result of analyzing the crystal structure of the catalyst through transmission electron microscopy (TEM) images, a structure in which nickel and copper metals were successfully supported on the Al₂O₃ and Al₂O₃_ZrO₂ supports was confirmed.

### Experimental Example 2: Analysis of Acidity of Catalyst According to Acid Ratio of Support

Catalysts were prepared using the method of Preparation Example 2, but by adjusting the ratio of Al₂O₃ and ZrO₂, and the results of confirming the acidity of the synthesized catalysts from the ammonia temperature-programmed desorption (NH₃-TPD) analysis results of FIG. 10 are shown in Table 3 below.

TABLE 3

| | Weak | Medium | Strong | |
|---|---|---|---|---|
| | acid NH₃ | acid NH₃ | acid NH₃ | Total |
| | (des) | (des) | (des) | acidity |
| | (mmol | (mmol | (mmol | (mmol |
| Catalyst | g⁻¹) | g⁻¹) | g⁻¹) | g⁻¹) |
| NiCu/Al₂O₃ | 0.063 | 0.371 | 0.430 | 0.864 |
| NiCu/Al₂O₃_{_{—}}ZrO₂ (10) | 0.039 | 0.274 | 0.534 | 0.847 |
| NiCu/Al₂O₃_{_{—}}ZrO₂ (7) | 0.068 | 0.348 | 0.476 | 0.892 |
| NiCu/Al₂O₃_{_{—}}ZrO₂ (5) | 0.057 | 0.378 | 0.487 | 0.922 |
| NiCu/Al₂O₃_{_{—}}ZrO₂ (3.5) | 0.021 | 0.140 | 0.519 | 0.680 |
| NiCu/Al₂O₃_{_{—}}ZrO₂ (1) | 0.002 | 0.052 | 0.330 | 0.384 |
| NiCu/ZrO₂ | 0.011 | 0.097 | 0.348 | 0.456 |

As a result of the experiment, it was confirmed that the total acidity decreased as the content of ZrO₂ in the support increased, and it was thus found that when the MTHF synthesis reaction was performed using each catalyst, it was possible to confirm the change in conversion rate according to acidity.

### Experimental Example 3: Analysis of MTHF Conversion Rate According to Reactant Ratio

The MTHF synthesis reaction was performed in a batch reactor using the catalyst prepared by the method of Preparation Example 1, but the reaction was performed by changing the composition of the reactants as shown in Table 4 below, and the conversion rate (%) and selectivity (%) were compared.

TABLE 4

| | | Reactant | | | | | | |
|---|---|---|---|---|---|---|---|---|
| Reaction | Catalyst | Conversion | FOL | THFOL | GVL | MF | 1,4-PDO | MTHF |
| Condition | (Powder) | rate | selectivity | selectivity | selectivity | selectivity | selectivity | selectivity |
| FUR | NiCu/Al₂O₃ | 100.0 | 10.0 | 56.0 | 7.00 | 2.00 | — | 25.0 |
| LA | NiCu/Al₂O₃ | 100.0 | — | — | — | — | — | 16.0 |
| FUR + LA(7:1) | NiCu/Al₂O₃ | 100 | | 43.0 | 14.0 | 4.00 | — | 39.0 |
| FUR + LA(4:1) | NiCu/Al₂O₃ | 100.0 | — | 25.0 | 15.0 | 5.00 | — | 55.0 |
| FUR + LA(3:2) | NiCu/Al₂O₃ | 100.0 | — | 22.0 | 28.0 | 4.00 | — | 46.0 |
| FUR + LA(1:1) | NiCu/Al₂O₃ | 100.0 | — | 16.0 | 42.0 | 6.00 | — | 36.0 |
| FUR + LA(2:3) | NiCu/Al₂O₃ | 100.0 | — | 13.0 | 50.0 | 4.00 | — | 33.0 |
| FUR + LA(1:4) | NiCu/Al₂O₃ | 100.0 | — | 17.0 | 54.0 | — | — | 29.0 |

As a result of the experiment, when furfural and levulinic acid were used in combination as reactants, the conversion rate was more than twice as high as when they were used alone. Accordingly, it was found that in the synthesis of MTHF, the reaction with furfural and the reaction with levulinic acid coexist and are both involved in the MTHF conversion process, thereby improving reaction efficiency.

In addition, as a result of experiments conducted by adjusting the ratio of furfural and levulinic acid in the reactants to 7:1, 4:1, 3:2, 1:1, 2:3, and 1:4, the MTHF conversion rate increased as the proportion of furfural increased up to 4:1, and when the proportion of furfural further increased to 7:1, the conversion rate decreased again.

From these results, it was confirmed that the predominant presence of furfural in the reactants prevents the formation of gamma-valerolactone (GVL) from levulinic acid, thereby improving the MTHF conversion rate, but when the amount of furfural is too large, this effect is somewhat reduced.

### Experimental Example 4: Analysis of MTHF Conversion Rate According to Catalyst Type

The MTHF synthesis reaction was performed in a batch reactor using the catalyst prepared by the method of Preparation Example 1, but the reaction was performed by changing the catalyst as shown in Table 5 below, and the conversion rate (%) and selectivity (%) were compared.

TABLE 5

| | | Reactant | | | | | | |
|---|---|---|---|---|---|---|---|---|
| Reaction | Catalyst | Conversion | FOL | THFOL | GVL | MF | 1,4-PDO | MTHF |
| Condition | (Powder) | rate | selectivity | selectivity | selectivity | selectivity | selectivity | selectivity |
| FUR + LA(4:1) | NiCu/Al₂O₃ | 100.0 | — | 25.0 | 15.0 | 5.00 | — | 55.0 |
| FUR + LA(4:1) | Ni/Al₂O₃ | 100.0 | — | 48.0 | 10.0 | 2.00 | — | 36.0 |
| FUR + LA(4:1) | Cu/Al₂O₃ | 100.0 | — | 33.0 | 18.0 | 21.0 | — | 28.0 |

As a result of performing the reaction using Ni/Al₂O₃ or Cu/Al₂O₃ instead of NiCu/Al₂O₃ as a catalyst with the ratio of furfural and levulinic acid in the reactants set to 4:1, when a catalyst having nickel or copper supported alone was used, the conversion rate to MTHF was 36.0% or 28.0%, respectively, but when a catalyst having both nickel and copper supported was used, it was confirmed that the conversion rate greatly increased to 55.0%.

### Experimental Example 5: Analysis of MTHF Conversion Rate According to Reaction Conditions

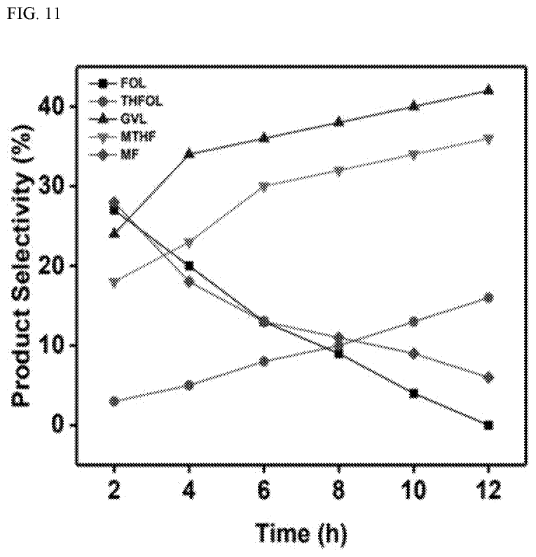

The MTHF synthesis reaction was performed in a batch reactor using the catalyst prepared by the method of Preparation Example 1, but the reaction was performed by changing the reaction pressure and temperature as shown in Table 6 below, and the conversion rate (%) and selectivity (%) were compared. In addition, the conversion rate according to time was confirmed under a condition in which the ratio of furfural and levulinic acid was 1:1, and the result graph is shown in FIG. 11.

TABLE 6

| | | Reactant | | | | | | |
|---|---|---|---|---|---|---|---|---|
| Reaction | Catalyst | Conversion | FOL | THFOL | GVL | MF | 1,4-PDO | MTHF |
| Condition | (Powder) | rate | selectivity | selectivity | selectivity | selectivity | selectivity | selectivity |
| FUR + LA (4:1) | NiCu/Al₂O₃ | 100.0 | — | 25.0 | 15.0 | 5.00 | — | 55.0 |
| FUR + LA(4:1) - | NiCu/Al₂O₃ | 100.0 | — | 37.0 | 8.00 | 35.0 | — | 20.0 |
| 10 bar | | | | | | | | |
| FUR + LA(4:1) - | NiCu/Al₂O₃ | 100.0 | — | 24.0 | 11.0 | — | — | 65.0 |
| 30 bar | | | | | | | | |
| FUR + LA(4:1) - | NiCu/Al₂O₃ | 100.0 | 16.0 | 37.0 | — | 15.0 | — | 27.0 |
| 150 °C | | | | | | | | |
| FUR + LA(4:1) - | NiCu/Al₂O₃ | 100.0 | — | 38.0 | — | — | — | 62.0 |
| 250 °C | | | | | | | | |

As a result of the above experiment, it was confirmed that when the existing reaction temperature (200 °C) was changed to 250 °C, the conversion rate to MTHF increased to 62.0%, and when the existing reaction pressure (hydrogen 20 bar) was increased to 30 bar, the conversion rate to MTHF increased to 65.0%. From these results, it was confirmed that an active reaction occurs under conditions of higher temperature and pressure.

In addition, as a result of comparing the conversion rate according to reaction time using furfural and levulinic acid at a ratio of 1:1, it was confirmed that as the reaction time increased, intermediates (furfuryl alcohol, methyl furan, etc.) decreased and the conversion rate to MTHF, which is the final target product, improved.

### Experimental Example 6: Analysis of MTHF Conversion Rate According to Reaction Solvent

The MTHF synthesis reaction was performed in a batch reactor using the catalyst prepared by the method of Preparation Example 1, but the reaction was performed by changing the reaction solvent as shown in Table 7 below, and the conversion rate (%) and selectivity (%) were compared.

TABLE 7

| | | Reactant | | | | | | |
|---|---|---|---|---|---|---|---|---|
| Reaction | Catalyst | Conversion | FOL | THFOL | GVL | MF | 1,4-PDO | MTHF |
| Condition | (Powder) | rate | selectivity | selectivity | selectivity | selectivity | selectivity | selectivity |
| FUR + LA(4:1) | NiCu/Al₂O₃ | 100.0 | — | 25.0 | 15.0 | 5.00 | — | 55.0 |
| FUR + LA(4:1) - | NiCu/Al₂O₃ | 100.0 | — | — | 50.0 | — | 32.0 | 18.0 |
| GVL | | | | | | | | |
| FUR + LA(4:1) - | NiCu/Al₂O₃ | 100.0 | — | 12.0 | 10.0 | — | 6.00 | 72.0 |
| Toluene | | | | | | | | |
| FUR + LA(4:1) - | NiCu/Al₂O₃ | 72.0 | — | 10.0 | 12.0 | — | 11.0 | 39.0 |
| CPME | | | | | | | | |
| FUR + LA(4:1) - | NiCu/Al₂O₃ | 50.0 | — | 7.00 | 8.00 | 4.00 | 9.00 | 22.0 |
| MIBK | | | | | | | | |

As a result of the experiment, when the reaction was performed using GVL, toluene, CPME, or MIBK instead of 2-propanol used as the existing reaction solvent, it was confirmed that the conversion rate to MTHF was the highest at 72% when toluene was used, and in the case of GVL, complete conversion to MTHF was not achieved under the relevant reaction conditions due to thermal stability.

### Experimental Example 7: Analysis of Conversion Rate According to Reaction Conditions

The MTHF synthesis reaction was performed in a batch reactor using the catalyst prepared by the sol-gel method of Preparation Example 2. The composition of the reactants and the type of catalyst were adjusted as shown in Table 8 below, and the conversion rate (%) and selectivity (%) were compared for each reaction.

TABLE 8

| | | Reactant | | | | | |
|---|---|---|---|---|---|---|---|
| Reaction | | Conversion | THFOL | GVL | MF | 1,4-PDO | MTHF |
| Condition | Catalyst (Powder) | rate | selectivity | selectivity | selectivity | selectivity | selectivity |
| FUR | NiCu/Al₂O₃ | 100.0 | 52.0 | 6.00 | — | — | 42.0 |
| FUR | NiCu/Al₂O₃_{_{—}}ZrO₂ (10) | 100.0 | 42.0 | 6.00 | — | 3.00 | 49.0 |
| FUR | NiCu/Al₂O₃_{_{—}}ZrO₂ (7) | 100.0 | 41.0 | 8.00 | — | 22.0 | 29.0 |
| FUR | NiCu/Al₂O₃_{_{—}}ZrO₂ (5) | 100.0 | 35.0 | 10.0 | — | 34.0 | 21.0 |
| FUR | NiCu/Al₂O₃_{_{—}}ZrO₂ (3.5) | 100.0 | 26.0 | 9.00 | — | 49.0 | 16.0 |
| FUR | NiCu/Al₂O₃_{_{—}}ZrO₂ (1) | 100.0 | 22.0 | 8.00 | — | 56.0 | 14.0 |
| FUR | NiCu/ZrO₂ | 100.0 | 17.0 | 6.00 | — | 65.0 | 12.0 |
| LA | NiCu/Al₂O₃ | 100.0 | — | 67.0 | — | — | 33.0 |
| LA | NiCu/Al₂O₃_{_{—}}ZrO₂ (10) | 100.0 | — | 59.0 | — | — | 41.0 |
| LA | NiCu/Al₂O₃_{_{—}}ZrO₂ (7) | 100.0 | — | 76.0 | — | — | 24.0 |
| LA | NiCu/Al₂O₃_{_{—}}ZrO₂ (5) | 100.0 | — | 82.0 | — | — | 18.0 |
| LA | NiCu/Al₂O₃_{_{—}}ZrO₂ (3.5) | 100.0 | — | 87.0 | — | — | 13.0 |
| LA | NiCu/Al₂O₃_{_{—}}ZrO₂ (1) | 100.0 | — | 90.0 | — | — | 10.0 |
| LA | NiCu/ZrO₂ | 100.0 | — | 92.0 | — | — | 8.00 |
| FUR + LA | NiCu/Al₂O₃ | 100.0 | 27.0 | 24.0 | 2.00 | — | 47.0 |
| (4:1) | | | | | | | |
| FUR + LA | NiCu/Al₂O₃_{_{—}}ZrO₂ (10) | 100.0 | 19.0 | 21.0 | 2.00 | 3.00 | 57.0 |
| (4:1) | | | | | | | |
| FUR + LA | NiCu/Al₂O₃_{_{—}}ZrO₂ (7) | 100.0 | 21.0 | 22.0 | 2.00 | 17.0 | 38.0 |
| (4:1) | | | | | | | |
| FUR + LA | NiCu/Al₂O₃_{_{—}}ZrO₂ (5) | 100.0 | 17.0 | 17.0 | 1.00 | 35.0 | 30.0 |
| (4:1) | | | | | | | |
| FUR + LA | NiCu/Al₂O₃_{_{—}}ZrO₂ (3.5) | 100.0 | 14.0 | 16.0 | 1.00 | 48.0 | 21.0 |
| (4:1) | | | | | | | |
| FUR + LA | NiCu/Al₂O₃_{_{—}}ZrO₂(1) | 100.0 | 12.0 | 13.0 | 2.00 | 53.0 | 20.0 |
| (4:1) | | | | | | | |
| FUR + LA | NiCu/ZrO₂ | 100.0 | 11.0 | 13.0 | — | 59.0 | 17.0 |
| (4:1) | | | | | | | |
| FUR + LA | NiCu/Al₂O₃ | 100.0 | 5.00 | 57.0 | — | — | 38.0 |
| (1:4) | | | | | | | |
| FUR + LA | NiCu/Al₂O₃_{_{—}}ZrO₂ (10) | 100.0 | 3.00 | 54.0 | — | — | 43.0 |
| (1:4) | | | | | | | |
| FUR + LA | NiCu/Al₂O₃_{_{—}}ZrO₂ (7) | 100.0 | 8.00 | 60.0 | — | — | 32.0 |
| (1:4) | | | | | | | |
| FUR + LA | NiCu/Al₂O₃_{_{—}}ZrO₂ (5) | 100.0 | 10.0 | 62.0 | — | 1.00 | 27.0 |
| (1:4) | | | | | | | |
| FUR + LA | NiCu/Al₂O₃_{_{—}}ZrO₂ (3.5) | 100.0 | 12.0 | 65.0 | — | 3.00 | 20.0 |
| (1:4) | | | | | | | |
| FUR + LA | NiCu/Al₂O₃_{_{—}}ZrO₂ (1) | 100.0 | 12.0 | 69.0 | — | 1.00 | 15.0 |
| (1:4) | | | | | | | |
| FUR + LA | NiCu/ZrO₂ | 100.0 | 12.0 | 72.0 | — | 5.00 | 11.0 |
| (1:4) | | | | | | | |

According to the ammonia temperature-programmed desorption analysis results of Experimental Example 2, it was confirmed that the acidity of the catalyst decreased as the content of ZrO₂ in the support increased. When these acidity experiment results were combined with the above reaction results, a tendency was confirmed that the conversion rate to MTHF increased as the acidity increased.

In addition, when furfural and levulinic acid were used in combination as reactants and the proportion of furfural was higher, the conversion rate to MTHF was the highest at 57.0%. Accordingly, it was confirmed that more effective conversion to MTHF was possible when a mixed form of furfural and levulinic acid was used as reactants in both the NiCu/Al₂O₃ catalyst synthesized by the wet impregnation method and the NiCu/Al₂O₃_ZrO₂ catalyst synthesized by the sol-gel method.

### Experimental Example 8: MTHF Synthesis Using Continuous Reactor

A reaction was performed in a continuous reactor for 12 hours under conditions of 250 °C and hydrogen 30 bar using the catalyst prepared according to Preparation Example 1 and furfural and levulinic acid at a ratio of 4:1 as reactants. For comparison, reactions were performed in a continuous reactor using furfural or levulinic acid alone as a reactant, and the conversion rate (%) and selectivity (%) of each reaction were compared and shown in Table 9 below.

TABLE 9

| | | Reactant | | | | | | |
|---|---|---|---|---|---|---|---|---|
| Reaction | Catalyst | Conversion | FOL | THFOL | GVL | MF | 1,4-PDO | MTHF |
| Condition | (Powder) | rate | selectivity | selectivity | selectivity | selectivity | selectivity | selectivity |
| FUR | NiCu/Al₂O₃ | 100.0 | 45.0 | 33.0 | 10.0 | — | — | 12.20 |
| LA | NiCu/Al₂O₃ | 87.0 | — | — | 79.0 | — | — | 21.0 |
| FUR+LA(4:1) | NiCu/Al₂O₃ | FUR: 100.0 | 30.0 | 37.7 | 7.00 | — | — | 26.0 |
| | | LA: 80.0 | | | | | | |

As a result of the reaction in the continuous reactor, the conversion rate to MTHF was lower than when the batch reactor was used, but it was confirmed that even in the continuous reactor, the conversion rate to MTHF was higher when a mixed form of reactants was used compared to furfural or levulinic acid used independently.

As specific parts of the content of the present invention have been described in detail above, it will be apparent to those skilled in the art that such specific description is merely a preferred embodiment and the scope of the present invention is not limited thereby. Therefore, the substantial scope of the present invention shall be defined by the appended claims and equivalents thereof.

What is claimed is:

1. A method for preparing 2-methyltetrahydrofuran, comprising obtaining 2-methyltetrahydrofuran (MTHF) by subjecting a furfural compound and a levulinic acid compound to hydrogenation and dehydration reactions.

2. The method for preparing 2-methyltetrahydrofuran according to claim 1, wherein a volume ratio of the furfural compound and the levulinic acid compound is 1:5 to 10:1.

3. The method for preparing 2-methyltetrahydrofuran according to claim 1, wherein the reaction is performed under a pressure of 10 to 50 bar.

4. The method for preparing 2-methyltetrahydrofuran according to claim 1, wherein the reaction is performed under a temperature condition of 150 to 400 °C.

5. The method for preparing 2-methyltetrahydrofuran according to claim 1, wherein a solvent used in the reaction comprises at least one selected from the group consisting of ethanol, propanol, butanol, toluene, cyclopentyl methyl ether (CPME), gamma-valerolactone (GVL), and methyl isobutyl ketone (MIBK).

6. The method for preparing 2-methyltetrahydrofuran according to claim 1, wherein the reaction is performed in the presence of a catalyst comprising an acid support, and nickel metal and copper metal supported on the acid support.

7. The method for preparing 2-methyltetrahydrofuran according to claim 1, wherein the reaction is performed in a batch reactor or a continuous reactor.

8. The method for preparing 2-methyltetrahydrofuran according to claim 1, wherein the method for preparing 2-methyltetrahydrofuran comprises
converting a part of levulinic acid into gamma-valerolactone in a lignocellulosic biomass comprising furfural and levulinic acid; and
obtaining 2-methyltetrahydrofuran by reacting the furfural and remaining levulinic acid.

9. A catalyst for preparing 2-methyltetrahydrofuran, comprising an acid support, and nickel metal and copper metal supported on the acid support.

10. The catalyst for preparing 2-methyltetrahydrofuran according to claim 9, wherein the acid support comprises at least one selected from the group consisting of aluminum oxide, zirconium oxide, zinc oxide, titanium oxide, magnesium oxide, cerium oxide, and lanthanum oxide.

11. A method for preparing a catalyst for preparing 2-methyltetrahydrofuran, comprising
mixing a nickel salt and a copper salt with an acid support and stirring; and
forming a catalyst in which nickel and copper are supported on the acid support by drying, calcining and reducing the mixture for which the stirring has been completed.

12. The method for preparing a catalyst for preparing 2-methyltetrahydrofuran according to claim 11, wherein the acid support is prepared through
forming a sol by stirring a metal salt solution and adding an acid;
obtaining a xerogel by drying the sol; and
obtaining an acid support by pulverizing and calcining the xerogel.

13. The method for preparing a catalyst for preparing 2-methyltetrahydrofuran according to claim 12, wherein the metal salt solution comprises aluminum alkoxide and zirconium alkoxide in a weight ratio of 1:1 to 10:1.

14. The method for preparing a catalyst for preparing 2-methyltetrahydrofuran according to claim 11, wherein the drying is performed at a temperature of 60 to 150 °C.

15. The method for preparing a catalyst for preparing 2-methyltetrahydrofuran according to claim 11, wherein the calcining is performed at a temperature of 350 to 600 °C.
